SEQUENCE LISTING (22 sequences)

SEQ ID NO: 1 - protein, 36 aa, synthetic construct
GAUUUAGACUACCCCAAAAACGAAGGGGACUAAAAC

SEQ ID NO: 2 - protein, 28 aa, synthetic construct
AUUAGGAUGAAGGAUAUAUUGGAGAAGA

SEQ ID NO: 3 - protein, 28 aa, synthetic construct
GGAAGCAGAUUUAGAUGUUUAGAAGGGA

SEQ ID NO: 4 - protein, 28 aa, synthetic construct
GUGAGGUUAUUAUAAGGUAGAGAUAUGA

SEQ ID NO: 5 - protein, 28 aa, synthetic construct
CAUGUGUGGGGAGUUCUUUGAUGGCAAC

SEQ ID NO: 6 - protein, 28 aa, synthetic construct
AUUAGUAGAAUAAUUAGUAGGUUUGAGA

SEQ ID NO: 7 - protein, 28 aa, synthetic construct
CAUUCGGUUGGGUGAUCUAGGCGGUGAC

SEQ ID NO: 8 - protein, 28 aa, synthetic construct
AUAAAAGGAAUGAAUUGUAUGAAUGUUG

SEQ ID NO: 9 - protein, 28 aa, synthetic construct
ACUGGAUUUGGCGUGCUGUUGAAAAGUU

SEQ ID NO: 10 - protein, 28 aa, synthetic construct
UGUGUCUCGAGAGGUGGGCUUGUUUUAA

SEQ ID NO: 11 - protein, 28 aa, synthetic construct
GAGUGGAUGGUAAUAUAGUAAAUGGAGU

SEQ ID NO: 12 - protein, 28 aa, synthetic construct
CAUGCGUGGGGAGUUCUUUGAUGGCAAC

SEQ ID NO: 13 - protein, 28 aa, unidentified
CAAGCUUUGCUACCGGCCUGAUAGAUUU

SEQ ID NO: 14 - protein, 28 aa, unidentified
CAAGCUUUGUUACCGGCCUGAUAGAUUU

SEQ ID NO: 15 - protein, 142 aa, synthetic construct
GAAATTAATACGACTCACTATAGGGAAGTCTAATCTCAAACCTTTTGAGAGAGATATTTCAACTGAAATCTATCAGGCCGGTAGCAAACCTTGTAATGGTGTTGAAGGTTTTAATTGTTACTTTCCTTTACAATCATATGGT

SEQ ID NO: 16 - protein, 142 aa, synthetic construct
GAAATTAATACGACTCACTATAGGGAAGTCTAATCTCAAACCTTTTGAGAGAGATATTTCAACTGAAATCTATCAGGCCGGTAACAAACCTTGTAATGGTGTTGCAGGTTTTAATTGTTACTTTCCTTTACGATCATATAGT

SEQ ID NO: 17 - protein, 28 aa, synthetic construct
AAATCTATCAGGCCGGTAGCAAACCTTG

SEQ ID NO: 18 - protein, 28 aa, synthetic construct
AAATCTATCAGGCCGGTAACAAACCTTG

SEQ ID NO: 19 - protein, 40 aa, synthetic construct
ATGATTGTAAAGGAAAGTAACAATTAAAACCTTCAACACC

SEQ ID NO: 20 - protein, 49 aa, synthetic construct
TAATACGACTCACTATAGGGCTCAAACCTTTTGAGAGAGATATTTCAAC

SEQ ID NO: 21 - protein, 40 aa, synthetic construct
ATGATCGTAAAGGAAAGTAACAATTAAAACCTGCAACACC

SEQ ID NO: 22 - protein, 49 aa, synthetic construct; identical to SEQ ID NO: 20

(19) United States
(12) Patent Application Publication
(10) Pub. No.: US 2025/0115951 A1
(43) Pub. Date: Apr. 10, 2025

(54) ENHANCED NUCLEIC ACID DETECTION USING CAS13 AND DESIGNED SECONDARY STRUCTURE

(71) Applicant: The Trustees of Princeton University, Princeton, NJ (US)

(72) Inventors: Cameron Myhrvold, Princeton, NJ (US); Benjamin Larsen, Hamilton, NJ (US); Ofer Kimchi, West Windsor, NJ (US); Owen Dunkley, Palo Alto, CA (US); Aartjan te Velthuis, Princeton, NJ (US)

(73) Assignee: The Trustees of Princeton University, Princeton, NJ (US)

(21) Appl. No.: 18/908,108

(22) Filed: Oct. 7, 2024

Related U.S. Application Data
(60) Provisional application No. 63/542,704, filed on Oct. 5, 2023.

(51) Int. Cl. C12Q 1/6823; C12N 9/22; C12N 15/11; C12Q 1/683; C12Q 1/6832

(52) U.S. Cl. CPC C12Q 1/6823; C12N 9/22; C12N 15/11; C12N 15/111; C12Q 1/6832; C12N 2310/20; C12Q 1/683

(57) ABSTRACT

Disclosed are techniques for enhanced nucleic acid detection using blocked or partially blocked crRNAs or target RNAs, and a composition of matter (detection reactions consisting of occluded crRNAs or target RNAs). Here, the mechanism of Cas 13 protein activation in response to RNA structure perturbations was systematically probed using a massively multiplexed screen. It was found that there are two distinct sequence-independent modes by which secondary structure affects Cas13 activity: structure in the protospacer region competes with the crRNA and can be disrupted via a strand-displacement mechanism, while structure in the region 3′ to the protospacer has an allosteric inhibitory effect. The kinetic nature of the strand displacement process was leveraged to improve Cas13-based RNA detection and enhance mismatch discrimination by up to 50-fold, enabling sequence-agnostic mutation identification at low (<1%) allele frequencies.

### CROSS-REFERENCE TO RELATED APPLICATIONS

The present application claims priority to U.S. Provisional Patent Application No. 63/542,704, filed Oct. 5, 2023, the contents of which are incorporated by reference herein in its entirety.

### STATEMENT REGARDING FEDERALLY SPONSORED RESEARCH OR DEVELOPMENT

This invention was made with government support under Grant Nos. R21 AI168808-01, DP2 AI175474-01, T32GM007388, and T32GM148739 awarded by the National Institutes of Health and Grant No. 75D30122C15113 awarded by the Centers for Disease Control. The government has certain rights in the invention.

### REFERENCE TO SEQUENCE LISTING

The instant application contains a Sequence Listing which has been submitted electronically in ST.26 XML file format, created on Oct. 3, 2024, is named PRIN97102_SL.xml and is 20,437 bytes in size. The ST.26 XML file is hereby incorporated by reference in its entirety.

### TECHNICAL FIELD

The present disclosure relates to techniques for enhanced nucleic acid detection, and specifically to techniques utilizing Cas13 in combination with secondary structures.

### BACKGROUND

The RNA-targeting CRISPR effector protein Cas13 holds tremendous promise for numerous applications, such as RNA targeting, detection, editing, and imaging. Cas13 is activated by the hybridization of a CRISPR RNA (crRNA) to a complementary single-stranded RNA (ssRNA) protospacer in a target RNA. Though Cas13 is not activated by double-stranded RNA (dsRNA) in vitro, it paradoxically demonstrates robust RNA targeting in environments where the vast majority of RNAs are highly structured. Understanding Cas13′s mechanism of binding and activation will be key to improving its ability to detect and perturb RNA; However, the mechanism by which Cas13 binds structured RNAs remains unknown.

### BRIEF SUMMARY

In various aspects, a method for enhanced nucleic acid detection may be provided. The method may include providing a DNA or RNA oligonucleotide complementary (either perfectly or partially complementary) to either a target RNA molecule or a crRNA spacer sequence. The crRNA spacer sequence may be a reverse compliment of a target region of the target RNA molecule.

The DNA or RNA oligonucleotides may have undergone various chemical modifications.

The method may include annealing the DNA or RNA oligonucleotide to the target RNA molecule or the crRNA spacer sequence by mixing the DNA or RNA oligonucleotide in excess with the target RNA molecule or the crRNA spacer sequence in an aqueous mixture at a first temperature (e.g., 85° C.). If a DNA oligonucleotide is used, a ratio of DNA:RNA in the aqueous mixture may be, e.g., 2:1-10:1. The aqueous mixture may include a salt, such as KCl.

The method may include ramping a temperature down from the first temperature to a second temperature (e.g., 4° C.) at a first rate. The method may include adding the aqueous mixture to one or more Cas13 RNA detection reagents. The Cas13 RNA detection reagents may be provided as an additional aqueous mixture. The additional aqueous mixture may include, e.g., water, an RNAse inhibitor, Leptotricia wadeii Cas13a (LwaCas13a), a detection buffer, a reporter RNA, crRNA, and magnesium acetate.

The method may include monitoring a fluorescent signal for a period of time (e.g., a period of time of 10 minutes-6 hours).

In various aspects, a composition of matter may be provided. The composition may include a Cas13 protein (such as Cas13, LwaCas13a (C2c2), LbuCas13a, PsmCas13b, PspCas13b, CcaCas13b, CasRx, Cas13d, orthologs thereof, or a combination thereof), a CRISPR RNA (crRNA), a target RNA molecule, a reporter RNA, and an occluder (such as a DNA oligonucleotide, a RNA oligonucleotide, a hairpin extension of the crRNA, or a combination thereof). The occluder may be of any appropriate length, such as 5 nt to 50 nt in length, 10 nt-40 nt in length, 15 nt-35 nt in length, or 20 nt-30 nt in length.

In various aspects, a method for improving crRNA design may be provided. The method may include using a strand displacement model of Cas13 reaction kinetics as a function of secondary structure to identify a target crRNA sequence that achieves desirable reaction kinetics. The method may then include producing crRNA having the target crRNA sequence.

### BRIEF DESCRIPTION OF THE FIGURES

FIG. 1 is a schematic illustration of a strand displacement reaction.

FIG. 2 is a flowchart of a method.

FIGS. 3-5 are graphs showing resulting fluorescent kinetic curves based on different amounts of structure being introduced into the target via intramolecular structure (unimolecular RNA) (3), RNA oligos (occluders) (4), and DNA occluders (5). Target input concentration: 7.5×10⁸ copies/μL.

FIG. 6 is a scatter plot comparing the impact of the different occlusion types depicted in FIGS. 3-5 on Cas13 activity; x axis: Cas13 activity when occluded by DNA oligos; y axis: Cas13 activity when occluded by intramolecular RNA or RNA occluders.

FIG. 7 is a graph showing Cas13 activity vs. occluder length for RNA occluders compared to two single-parameter models: an equilibrium model based on crRNA-target hybridization free energies (dotted line) and a free-energy-independent strand displacement model (dashed line). Effects of changing the single parameter are indicated by arrows. The thick solid line is NTC.

FIG. 8 is a schematic illustration showing an overview of the multiplexed assay, in which a total of 4,608 simultaneous assays were performed, with oligo occluders of lengths 10, 14, 21, 28 tiling each protospacer region in 3 nt increments.

FIG. 9 is a plot showing an overview of the Cas13 activity data from the multiplexed assay. Each data point represents the mean activity resulting from averaging four time series curves, normalized to the non-occluded condition; positive and negative controls are not shown.

FIG. 10 is a cumulative histogram of inhibition asymmetry, defined as the ratio of activities when the same numbers of nucleotides are occluded at the 3′ vs. 5′ ends of the protospacer.

FIGS. 11 and 12 are plot showing normalized Cas13 activity for 21mer (11) and 28mer (12) occluders with different start positions in the region around the protospacer.

FIG. 13 is a plot showing strand displacement model prediction for activity as a function of occluding oligo, with the fill of the circles indicating the fraction of seed or switch regions occluded by the oligo.

FIG. 14 is a bar chart showing the inhibitory effect of 28mer occluders overlapping the protospacer or the region 3′ to the protospacer, at different occlude concentrations and when annealing the occluder before or at the same time as the crRNA.

FIGS. 15 and 16 are schematic illustrations showing strand displacement by Cas13 with a perfectly-matched target sequence (15) versus one containing a mismatch (16).

FIG. 17 is a graph showing ODE-based model predictions of crRNA/target hybridization kinetics with and without occlusion and mismatches.

FIG. 18 is a graph of kinetic curves showing detection of a target sequence with and without a single A>U mismatch at spacer position 5, in the presence and absence of occlusion.

FIG. 19 is a bar chart of maximum fluorescence ratios with and without occlusion at a variety of target input concentrations.

FIG. 20 includes violin plots showing the ability of Cas13 to distinguish between wildtype targets and targets containing mutations at four different positions in the protospacer both with and without occlusion; position is relative to the 5′ end of the protospacer. Each data point is the discrimination ratio of perfect-match to mismatched sequence.

FIG. 21 is a graph showing data from FIG. 20, but organized by mutation type.

FIG. 22 is a heatmap showing the ability of Cas13 to detect spiked-in target in a background of mismatched sequence at decreasing allele frequencies, both with and without occlusion. Asterisks indicate statistically significant detection over the no-spike in control. Significance determined by one-tailed t-test p<0.05. Activity discrimination is defined analogously to mismatch discrimination.

FIG. 23 is a specificity matrix showing Cas13 activity normalized for each target to its corresponding crRNA, with and without occlusion, for all possible crRNA and target nucleotides at position 5.

FIG. 24 is a schematic of detection workflow.

FIG. 25 is a chart showing detection of Delta and Omicron SARS-CoV-2 spike gene RNA from amplified viral seedstocks. Fluorescence at the timepoint corresponding to the maximum discrimination ratio is shown, normalized independently for each target to its maximum.

FIG. 26 is a chart showing Cas13-based discrimination of ancestral (627E) Influenza A virus (IAV) variant from multiple mammalian-adapted (627K) strains, as well as the rare 627V variant, all distinguished by a single-nucleotide substitution. Each target's final fluorescence at 180 minutes was normalized independently to its maximum.

FIG. 27 is a chart showing discrimination of a single-nucleotide substitution in IAV strains conferring oseltamivir resistance in 6 different isolates, using a single guide pair per NA subtype. Each target's final fluorescence at 180 minutes was normalized independently to its maximum.

FIG. 28 is a plot showing discrimination of USA patient samples infected with Delta and Omicron strains of SARS-CoV-2. Final fluorescence (x-axis) is used to distinguish positive from negative calls; maximum fluorescence ratio (y-axis) is used to distinguish variants. “-”: not detected.

FIG. 29 is a plot showing discrimination of 627E from K in samples from UK patients infected with seasonal IAV. Final fluorescence (x-axis) is used to distinguish positive from negative calls; maximum fluorescence ratio (y-axis) is used to distinguish variants. “-”: not detected.

FIG. 30 is a plot showing discrimination of Dutch patient samples infected with oseltamivir-sensitive (H) and resistant (Y) single-nucleotide IAV variants. Final fluorescence (x-axis) is used to distinguish positive from negative calls; maximum fluorescence ratio (y-axis) is used to distinguish variants. “-”: not detected.

FIG. 31 is a chart showing discrimination of IAV 627E from 627K variants in patient samples and clinical isolates from patients who have tested positive for H5N1 since 2023 in Cambodia. Fluorescence at the timepoint corresponding to the maximum discrimination ratio was normalized independently for each target to its maximum.

FIG. 32 is a plot showing some results for using occluded Cas13 to distinguish 7 variants of codon 12 of the KRAS gene with mCARMEN. Final fluorescence at 180 minutes was normalized independently for each target to its maximum.

### DETAILED DESCRIPTION

As used herein, the term “about [a number]” refers to a range that is ±10%. Preferably ±5%, more preferably ±2%, still more preferably ±1% of the number, and most preferably the number itself. For example, “about 10” is typically 9-11 (±10%), preferably 9.5-10.5, more preferably 9.8-10.2, still more preferably 9.9-10.1, and most preferably 10.

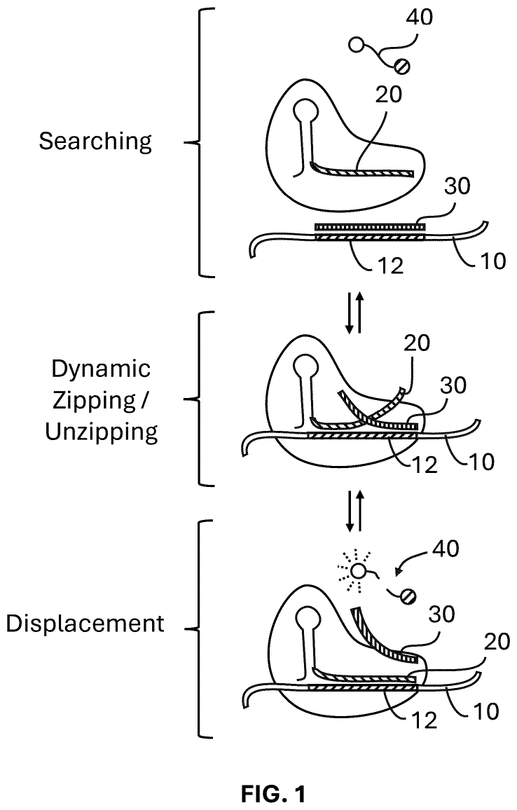

As used herein, the term “occluder” refers to a secondary structure that can be added to an ssRNA protospacer. Here, an ssRNA protospacer sequence was designed to which one may add variable amounts of secondary structure by either intramolecular extension of an RNA hairpin, or by adding external complementary RNA or DNA oligonucleotides of different lengths, termed “occluders” (FIG. 1A).

The disclosed approach leverages the kinetic nature of strand displacement reactions in an enzymatic context to enhance specificity and mismatch detection of kinetic Cas13 assays. Briefly, referring to FIG. 1, strand displacement reaction may involve several steps. An initial searching step may occur where crRNA (20) “searches” for a target (12) on a nucleotide sequence (10) to bind to. The target is generally coupled to an occluder (30) in this step. After initial binding to a part of the target (12), the crRNA (20) and occluder (30) undergo a random walk process (the “dynamic zipping/unzipping” step) until one or the other is fully displaced. As shown in FIG. 1, in the “displacement” step, displacement of the occluder (30) leads to Cas13 activation. A fluorescent RNA (40) can be used to report activity.

Cas13 mismatch detection as measured by fluorescence ratios may be increased by a factor of ˜50. Cas13 mismatch detection across a range of mutations is increased from ˜77% accuracy to ˜100% accuracy with no sample manipulation. The disclosed method provides robust, comprehensive, and sequence-agnostic mismatch detection and target specificity with no sample manipulation.

The inventors have found that there are two sequence-independent modes by which secondary structure affects Cas13 activity: in the protospacer, structure competes with the crRNA and can be disrupted via a strand-displacement mechanism, while 3′ to the protospacer, structure has an allosteric inhibitory effect. The kinetic nature of strand displacement can be leveraged to improve Cas13-based RNA detection, enhancing mismatch discrimination by up to 50-fold and enabling sequence-agnostic mutation identification at low (<1%) allele frequencies. The technique is flexible, and useful for various applications. For example, using this method, which is referred to herein as “occluded Cas13”, human-adaptive mutations can be identified in SARS-CoV-2 and human and avian influenza A viruses, as well as oseltamivir-resistance mutations in influenza A virus. The assay was deployed on 69 patient samples and 11 clinical isolates from 4 countries, including samples from the 2023-4 H5N1 avian flu outbreak and 2016-9 seasonal influenza epidemics, finding robust detection and variant discrimination.

This sets a new standard for CRISPR-based nucleic acid detection and enables intelligent and secondary-structure-guided target selection while also expanding the range of RNAs available for targeting with Cas13.

Disclosed herein are enhanced nucleic acid detection using Cas13 and designed secondary structure. More particularly, disclosed is a method for enhanced nucleic acid detection using blocked or partially blocked crRNAs or target RNAs, and a composition of matter (detection reactions consisting of occluded crRNAs or target RNAs).

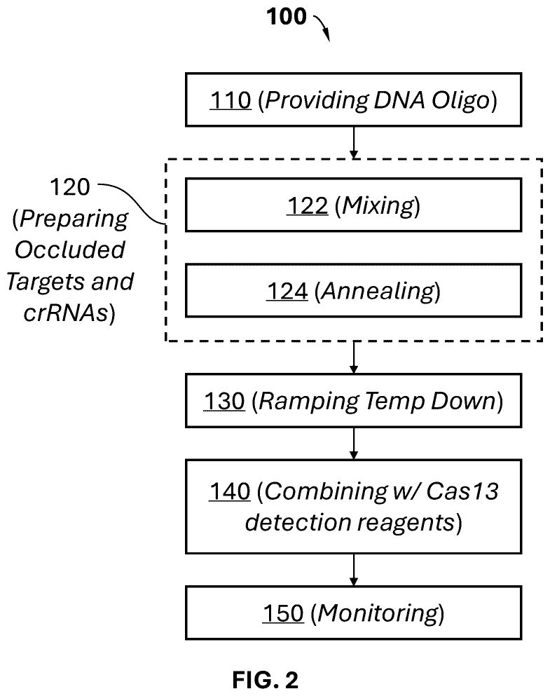

A method for enhanced nucleic acid detection may be provided. Referring to FIG. 2, the method (100) may include providing (110) a DNA oligonucleotide complementary to either a target RNA molecule or a crRNA spacer sequence. The crRNA spacer sequence may be a reverse compliment of the target RNA molecule.

The method may include preparing (120) an occluded target RNA molecule and/or crRNA spacer sequence. This may include mixing (122) a DNA oligonucleotide in excess with the target RNA molecule or the crRNA spacer sequence, as appropriate, in an aqueous mixture. In some embodiments, the ratio of DNA:RNA in the aqueous mixture may be, e.g., at least 1.01:1, 2:1, 3:1, 4:1, or 5:1, up to 10:1, 15:1, 20:1, 50:1, or 100:1, including any combination or subranges thereof. In one preferred embodiment, the ratio may be 2:1-10:1.

This may include annealing (124) the DNA oligonucleotide at an annealing temperature for a period of time (an “annealing time”). The annealing temperature may be any appropriate annealing temperature for the DNA oligonucleotide and the target RNA molecule or the crRNA spacer sequence. The annealing temperature may be, e.g., at least 60° C., 65° C., 70° C., 75° C., or 80° C., up to 85° C., 90° C., or 95° C., including any combination or subranges thereof. In one preferred embodiment, the annealing temperature may be 80° C.-90° C., such as about 85° C. The annealing time may be any appropriate time for enabling the annealing to occur. The annealing time may be, e.g., at least 30 seconds, at least 1 minute, or at least 2 minutes up to 5 minutes, 10 minutes, or 30 minutes. In one preferred embodiment, the annealing time may be 2 minutes-5 minutes, such as about 3 minutes.

The aqueous mixture may include a salt, such as NaCl, KCl, MgCl₂, and CaCl₂. In some embodiments, the aqueous mixture may be free of a salt. The aqueous mixture may include a buffer solution, such as Tris base or Tris-HCl. In some embodiments, the aqueous mixture may be free of a buffer solution.

The method may include ramping (130) a temperature down from the annealing temperature to a target temperature at a target cooling rate. The target temperature may be any appropriate temperature. The final temperature to which the mixture is cooled may be a temperature that is below room temperature and above a freezing temperature of the aqueous mixture. In some embodiments, the final target temperature may be a temperature that is at least 1° C., 2° C., 3° C., or 4° C., up to 10° C., 11° C., 12° C., 13° C., 14° C., or 15° C., including any combination or subranges thereof. In one preferred embodiment, the temperature may be 2° C.-10° C., such as about 4° C.

The target rate may be any appropriate rate. In a preferred embodiment, the rate is constant. In some embodiments, the rate may vary over time during the ramping step (for example, it may cool slowly at first and speed up as temperature drops, or the reverse, where it cools quickly at first and slows down as temperature drops). In some embodiments, there may be two phases of cooling, such as an initial phase from the first temperature to an intermediate temperature at a controlled rate, and a second phase from the intermediate temperature the second temperature at a different rate or an uncontrolled rate.

In some embodiments, the target rate may be a rate that is at least 0.01° C./min, at least 0.05° C./min, or at least 0.1° C./min up to 0.5° C./min, 1° C./min, or 2° C./min, including any combination or subranges thereof. In one preferred embodiment, the temperature may be 0.05° C./min-0.5° C./min, such as about 0.1° C./min.

The method may include combining (140) the aqueous mixture and one or more Cas13 RNA detection reagents. This may any appropriate means of combining, including, e.g., adding the aqueous mixture to a detection mixture, adding the Cas13 RNA detection reagents to the aqueous mixture, or some variation thereof.

The Cas13 RNA detection reagents may be provided as an additional aqueous mixture (e.g., a detection mixture). The detection mixture may include, e.g., water, an RNAse inhibitor, a Cas13 protein (such as Leptotricia wadeii Cas13a (LwaCas13a)), a buffer (such as a Tris HCl buffer), a detection buffer, a reporter RNA (preferably a fluorescent reporter), crRNA, and magnesium acetate. The detection buffer may include, e.g., a biological buffer (such as (4-(2-hydroxyethyl)-1-piperazineethanesulfonic acid) (HEPES)), a salt (such as NaCl or KCl), a polyethylene glycol (such as PEG-8000) and water (such as nuclease-free water)).

The Cas13 protein may be present in any appropriate amount. In various embodiments, the Cas13 protein may be present in an amount of at least 1 nM, 5 nM, 10 nM, 20 nM, 30 nM, 40 nM, 50 nM, or 60 nM, up to 60 nM, 70 nM, 80 nM, 90 nM, 100 nM, or 1 μM, including any combination or subranges thereof. In one embodiment, the Cas13 protein may be present in a total amount of 5 nM-100 nM, such as about 10 nM, about 45 nM or about 90 nM.

The crRNAs may be pre-annealed to DNA occluders as disclosed herein. In preferred embodiments, the crRNAs may be present in a total amount less than the amount of the Cas13 protein. The crRNAs may be present in a total amount of at least 0.1 nM, 1 nM, 2 nM, 3 nM, 4 nM, 5 nM, 10 nM, or 20 nM, up to 60 nM, 70 nM, 80 nM, 90 nM, 100 nM, 500 nM, or 1 μM, including any combination or subranges thereof. In one preferred embodiment, the crRNAs may be present in an amount of 3 nM to 60 nM, such as about 5 nM, about 22.5 nM, or about 45 nM.

Any appropriate RNAse inhibitor may be utilized. In one embodiment, the RNAse inhibitor is a murine RNAse inhibitor. The RNAse inhibitor may be present in any appropriate amount. The RNAse inhibitor may be present in a total amount of at least 0.1 U/μL, 0.25 U/μL, 0.5 U/μL, or 1 U/μL up to 2 U/μL, 3 U/μL, 4 U/μL, 5 U/μL, or 10 U/μL, including any combination or subranges thereof. In one embodiment, the RNAse inhibitor may be present in a total amount of 0.5 U/μL-2 U/μL, such as about 1 U/μL.

Any reporter RNA appropriate for the intended means of detecting may be provided. For example, a fluorescent reporter may be utilized. The fluorescent reporter may include a cell-permeable small-molecule fluorogenic dye (e.g., a fluorogen, such as 5-carboxyfluorescein (5FAM) specifically bound to an RNA structure. The reporter RNA may include, e.g., 5FAM The reporter RNA may be present in any appropriate amount. The reporter RNA may be present in a total amount of at least 1 nM, 5 nM, 10 nM, 20 nM, 30 nM, 40 nM, 50 nM, or 60 nM, up to 60 nM, 70 nM, 80 nM, 90 nM, 100 nM, or 1 μM, including any combination or subranges thereof. In one preferred embodiment, the reporter RNA may be present in an amount of 10 nM to 90 nM, such as about 60 nM.

In some embodiments, The aqueous mixture may be combined with a detection mixture in an amount such that the v/v % of the aqueous mixture is at least 1%, at least 2%, at least 3%, at least 4%, at least 5%, at least 6%, at least 7%, at least 8%, at least 9%, or at least 10% up to 15%, 20%, 25%, 30%, 35%, 40%, 45%, or 50%, including any combination or subranges thereof. In one preferred embodiment, the aqueous mixture is present in the combination in an amount of 5% v/v-15% v/v, such as about 10% v/v (e.g., the combination is about 10% v/v aqueous mixture with about 90% detection mixture).

The method may include monitoring (150) a fluorescent signal for a period of time (the “monitoring time”). The monitoring time may be, e.g., at least 1 minute, at least 5 minutes, at least 10 minutes, at least 30 minutes, or at least 1 hour, up to 1 hour, 2 hours, 3 hours, 4 hours, 5 hours, 6 hours, 7 hours, 8 hours, 9 hours, or 10 hours. In one preferred embodiment, the monitoring time may be at least 10 minutes. In one preferred embodiment, the monitoring time may be no more than 6 hours. The monitoring may occur via any appropriate analysis technique or equipment (e.g., if a fluorescent reporter is used, means for detecting and recording fluorescent intensities, such as with a plate reader, may be appropriate).

In various aspects, a composition of matter may be provided. The composition may include a Cas13 protein, a CRISPR RNA (crRNA), a target RNA molecule, a reporter RNA, and an occluder.

Any appropriate Cas13 protein or variant thereof may be utilized here. Such proteins are well known in the art. Non-limiting examples of Cas13 proteins include Cas13, LwaCas13a (C2c2), LbuCas13a, PsmCas13b, PspCas13b, CcaCas13b, CasRx, Cas13d, or orthologs thereof (or a combination thereof).

Any appropriate occlude may be utilized here. The occlude may be, e.g., a DNA oligonucleotide, a RNA oligonucleotide, a hairpin extension of the crRNA, or a combination thereof.

In various aspects, a method for improving crRNA design may be provided. The method may include using a strand displacement model of Cas13 reaction kinetics as a function of secondary structure to identify a target crRNA sequence that achieves desirable reaction kinetics. The method may then include producing crRNA having the target crRNA sequence.

### EXAMPLES

### RNA Structure Reduces LwaCas13a Activity

To isolate the effect of RNA structure on Cas13 activity, an ssRNA protospacer sequence was designed to which variable amounts of secondary structure could be added by either intramolecular extension of an RNA hairpin, or by adding external complementary RNA or DNA oligonucleotides of different lengths, termed “occluders”. In some embodiments, the occluders are perfectly complementary. In some embodiments, the occluders are only partially complementary. In some embodiments, only one mismatch is present (e.g., either a single nucleotide mismatch, or an insertion or deletion mismatch). In some embodiments, two mismatches may be present.

Occluders may be of any length, but preferably the length is a length of about 5 nt to about 50 nt, more preferably from about 10 nt to about 40 nt, even more preferably about 15 nt to about 35 nt, and still more preferably about 20 nt to about 30 nt. In some preferred embodiments, the length is at least 5 nt, 10 nt, 15 nt, or 20 nt in length up to 30 nt, 35 nt, 40 nt, 45 nt, or 50 nt, including all subranges and combinations thereof.

In some embodiments, the DNA or RNA oligonucleotides may have undergone chemical modification. Examples of chemical modification include phosphorothioate modifications, 2′-O-Methyl RNA modifications, locked nucleic acids (LNA) modification, morpholino oligonucleotides modification, peptide nucleic acids (PNA) modification, and/or capping modifications.

The protospacer was designed to reflect the viral sequence diversity used to train ADAPT (Metsky, H. C. et al., “Designing sensitive viral diagnostics with machine learning”, Nat. Biotechnol. 40, (2022)) and to have minimal secondary structure.

Specifically, an RNA molecule of length 961 nucleotides was designed with minimal internal secondary structure. After an initial G nucleotide, the molecule is comprised of 10 target blocks, each defined by a 34 nt buffer region, a 28 nt protospacer, and a second 34 nt buffer region. For this design, it was sought to have as many as possible of the 28 nt protospacers resemble natural sequences.

To this end, the design process started with a set of 18,508 28-nt-long protospacer sequences compiled from the ADAPT dataset, which has a sequence composition representative of viral diversity. 3,391 sequences with poly-A, poly-C, or poly-U stretches ≥5 nts or poly-G stretches ≥4 nts were removed. Of the remaining sequences, 6,459 were removed which had low average measured activity, defined as (out_log_k)≤−2 (on a logarithmic scale from −4 to 0, where 0 is high activity) using the activity definitions and measurements from Metsky. LandscapeFold48 was used with parameter m=2 (m represents the minimum allowed stem length), disallowing pseudoknots, to predict the structure landscapes of the remaining sequences. LandscapeFold predicted that 1,287 of these remaining sequences had extremely low intramolecular structure, defined as all nucleotides having a ≥40% probability of being unpaired in equilibrium.

The design process then aimed to find a set of these sequences that were all dissimilar from one another. First, given a sequence s, we found all those sequences with a Hamming distance ≤15 from s. (A pair of sequences with a Hamming distance of h share all but h nucleotides). Of these sequences, the one with the least secondary structure was chosen to keep and removed the others, with total amount of secondary structure quantified as Σnpn where the sum is over nucleotides and pn is the probability of the nucleotide being paired in equilibrium. This step was repeated for each sequence s not already removed. Next, a Smith-Waterman alignment was used to check for sequence similarity in non-identical nucleotide positions, repeating the same procedure as above but, instead of Hamming distance, using the criteria of an alignment score ≥9 to define sequence similarity, where the alignment score parameters were (+1, −2, −2) for (match, mismatch, gap). This procedure resulted in a set of 20 sequences all distant from one another in sequence space.

Finally, although the design process involved ensuring each of these sequences had low secondary structure, it was also desired to minimize binding between these sequences. For each pair of sequences, LandscapeFold was used with parameter m=3 to predict the structure of the two strands, allowing for both intra- and inter-molecular interactions. Two sequences were defined to be incompatible if the resulting prediction had any nucleotide on either sequence with a ≤40% probability of being unpaired in equilibrium. The possible ordered sets of mutually compatible sequences were exhaustively enumerated, finding 60 ordered sets of 5 mutually compatible sequences, and no set of 6 mutually compatible sequences. Of these 60 sets, the one with the least structure was chosen. Under the assumption that entropic loop closure costs will create a barrier to non-neighbor sequence pairing (i.e. that each sequence is less likely to pair to a sequence that isn't its neighbor), structure were defined here as the sum, over the 4 pairs of neighboring sequences, of the maximum probability of a nucleotide being paired in that sequence pair. Thus, the design process arrived at a set of 5 distinct sequences from ADAPT with minimal intra- and inter-molecular structure.

These 5 sequences became the protospacer sequences corresponding to crRNAs 2, 4, 6, 8, 9 (see Table 1, below).

TABLE 1
(crRNA names, direct repeat sequences, and spacer sequences)

| crRNA | | |
|---|---|---|
| name | Direct repeat sequence | Spacer sequence |
| cr1 | GAUUUAGACUACCCCAAAAACGAA | AUUAGGAUGAAGGAUAUA |
| | GGGGACUAAAAC (SEQ ID NO. 1) | UUGGAGAAGA (SEQ ID NO. 2) |
| cr2 | GAUUUAGACUACCCCAAAAACGAA | GGAAGCAGAUUUAGAUGU |
| | GGGGACUAAAAC (SEQ ID NO. 1) | UUAGAAGGGA (SEQ ID NO. 3) |
| cr3 | GAUUUAGACUACCCCAAAAACGAA | GUGAGGUUAUUAUAAGGU |
| | GGGGACUAAAAC (SEQ ID NO. 1) | AGAGAUAUGA (SEQ ID NO. 4) |
| cr4 | GAUUUAGACUACCCCAAAAACGAA | CAUGUGUGGGGAGUUCUUU |
| | GGGGACUAAAAC (SEQ ID NO. 1) | GAUGGCAAC (SEQ ID NO. 5) |
| cr5 | GAUUUAGACUACCCCAAAAACGAA | AUUAGUAGAAUAAUUAGU |
| | GGGGACUAAAAC (SEQ ID NO. 1) | AGGUUUGAGA (SEQ ID NO. 6) |
| cr6 | GAUUUAGACUACCCCAAAAACGAA | CAUUCGGUUGGGUGAUCUA |
| | GGGGACUAAAAC (SEQ ID NO. 1) | GGCGGUGAC (SEQ ID NO. 7) |
| cr7 | GAUUUAGACUACCCCAAAAACGAA | AUAAAAGGAAUGAAUUGU |
| | GGGGACUAAAAC (SEQ ID NO. 1) | AUGAAUGUUG (SEQ ID NO. 8) |
| cr8 | GAUUUAGACUACCCCAAAAACGAA | ACUGGAUUUGGCGUGCUGU |
| | GGGGACUAAAAC (SEQ ID NO. 1) | UGAAAAGUU (SEQ ID NO. 9) |
| cr9 | GAUUUAGACUACCCCAAAAACGAA | UGUGUCUCGAGAGGUGGGC |
| | GGGGACUAAAAC (SEQ ID NO. 1) | UUGUUUUAA (SEQ ID NO. 10) |
| cr10 | GAUUUAGACUACCCCAAAAACGAA | GAGUGGAUGGUAAUAUAG |
| | GGGGACUAAAAC (SEQ ID NO. 1) | UAAAUGGAGU (SEQ ID NO. 11) |

The other 5 protospacer sequences as well as the buffer regions were compiled out of 64 16-nt-long DNA sequences with minimal internal structure from Shortreed et al. Seven of these sequences with poly-A or poly-T stretches ≥5 nts were removed. Concatenating these sequences resulted in a long sequence with minimal structure, which we used to construct the rest of the 961 nt-long RNA target. NUPACK 3 was used to predict the structure of the resulting target, finding various predicted stems. Individual point mutations were then made by hand in the buffer regions and non-ADAPT-derived protospacers to minimize the probabilities of the resulting stems (ensuring NUPACK 3 predicted no base pair forming with probability ≥60% in equilibrium), as well as to remove sequence similarity between targets (ensuring there are no more than 5 identical consecutive nucleotides between the protospacer regions, no more than 6 identical consecutive nucleotides between two regions spanning a protospacer and a buffer, and no more than 8 identical consecutive nucleotides in buffer regions).

Finally, a “shuffled” version of the target was created, placing the target blocks (numbered 1-10 from 5′ to 3′ in the original target) in the following order: 1, 4, 2, 7, 5, 3, 9, 6, 8, 10. It was ensured that NUPACK 3 did not predict any base pair forming with probability ≥60% in the resulting sequence.

For some initial experiments, the ADAPT dataset sequences were filtered to those with high activity ((out_log_k)>−2) and perfect complementarity between target and crRNA in the ADAPT dataset. LandscapeFold's prediction of the secondary structure of each candidate protospacer sequence was then measured. For each nucleotide, the total probability that the nucleotide is unpaired in equilibrium was calculated. The protospacer chosen had each nucleotide with at least a 92% probability of being unpaired in equilibrium.

### Cas13 Detection Assays

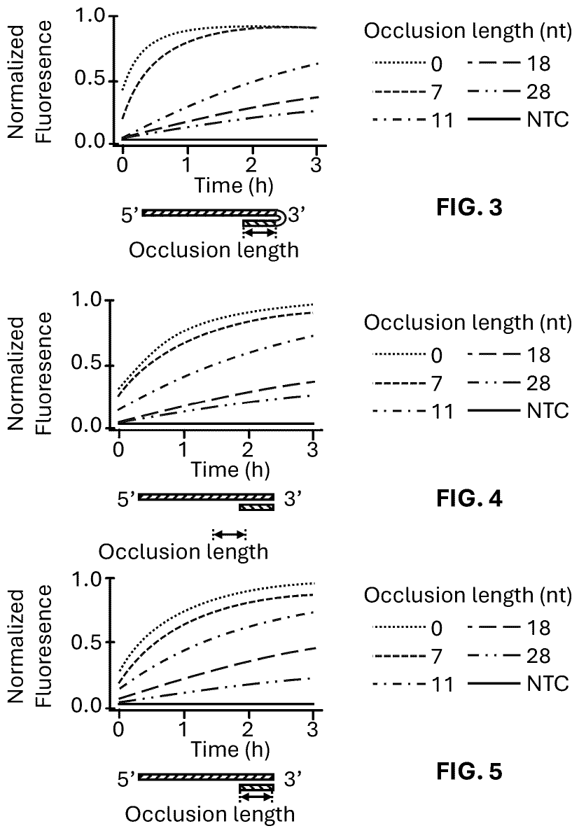

Standard bulk detection assays were performed by mixing target RNA or cDNA at a ratio of 10% v/v with 90% Cas13 detection mix. The detection mix consisted of 1×RNA Detection Buffer (20 mM HEPES pH 8.0, 54 mM KCl, 3.5% PEG-8000 in NF water), supplemented with 45 nM purified LwaCas13a (Genscript, stored in 100 mM Tris HCl pH 7.5 and 1 mM DTT), 1 U/μL murine RNAse Inhibitor (New England Biolabs), 62.5 nM fluorescent reporter (/5FAM/rUrUrUrUrUrU/IABkFQ/from IDT), 22.5 nM processed crRNA (IDT), and 14 mM MgOAc. In experiments using crRNA occlusion, crRNAs were pre-annealed to DNA occluders as described herein, and used at a final concentration of 22.5 nM. Experiments using cDNA as the input included 0.3 mM rNTPS (New England Biolabs) and 1 u/uL T7 polymerase (Biosearch). Minor adjustments to the detection mix were made for experiments using other orthologs of Cas13; for RfxCas13d, the Cas13 concentration was set to 90 nM and crRNA concentration was set to 45 nM. For LbuCas13a, Cas13 concentration was set to 10 nM and crRNA concentration to 5 nM. 15 μL reactions were loaded in technical triplicate (see FIGS. 3-7) or duplicate (see FIG. 14, 19-23) onto a Greiner 384 well clear-bottom microplate (item no. 788096) and measured on an Agilent BioTek Cytation 5 or Synergy H1 microplate reader for 3 hours with excitation at 485 nm and detection at 528 nm every five minutes.

For tiling assays and mCARMEN KRAS assay, Standard Biotools genotyping IFC (192.24 format) was used in a BioMark HD for multiplexed detection. Assay mix (10% of final reaction volume) contained 1×Assay Detection Mix (Standard Biotools) supplemented with 100 nM crRNA, 100 nM LwaCas13a (Genscript, stored in 100 mM Tris HCl pH 7.5 and 1 mM DTT). Sample mix (90% of final reaction volume) contained 1×Sample Buffer (44 mM Tris-HCl pH 7.5, 5.6 mM NaCl, 10 mM (tiling experiment) or 2 or 14 mM (KRAS) MgCl, 1.1 mM DTT, 1.1% w/v PEG-8000), supplemented with murine RNAse Inhibitor (1 U/μL, NEB), fluorescent reporter (500 nM, IDT), 1×ROX reference dye (used for normalization of random fluctuations in fluorescence between chambers) (Standard Biotools), 1×GE Buffer (Standard Biotools), 20 mM KCl, and occluded RNA target (9×108 cp/μL). Experiments using cDNA as the input included 0.9 mM rNTPS (New England Biolabs) and 0.125 U/uL T7 polymerase (Biosearch). Sample volumes of 3.5 μL and assay volumes of 3.5 μL, in addition to appropriate volumes of Control Line Fluid, Actuation Fluid, and Pressure Fluid (Standard Biotools), were loaded onto 192.24 genotyping IFC chip (Standard Biotools). Chips were then placed into the Fluidigm Controller and loaded and mixed using the Load Mix 192.24 GE script (Standard Biotools). After mixing, reactions (four technical replicates each) were run on BioMark HD at 37° C. for 8 hours with measurements taken in the fluorescein amidite (FAM) and the carboxyrhodamine (ROX) channels every five minutes. Normalized and background-subtracted fluorescence for a given time point was calculated as (FAM-FAM_background)/(ROX-ROX_background) where FAM_background and ROX_background are the FAM and ROX background measurements.

For fluorescent in-tube detection assays, Cas13 detection mix was prepared as in bulk detection assays with fluorescent reporter raised to 250 nM and crRNA raised to 45 nM in the final reaction. 33 uL reactions were incubated at 37° C. for 3 hours. Every 30 minutes reactions were visualized with UV light on a transilluminator and captured with a smartphone camera.

The ability of the structured protospacers to activate LwaCas13a was tested using cleavage of a quenched fluorescent RNA to report activity. Increased secondary structure decreased Cas13 activity across all three assay conditions. See FIGS. 3-5.

Cas13 activity was then quantified by fitting the fluorescence curves to effectively first-order reaction equations, defining activity as the rate of reporter cleavage (hr⁻¹), a proxy for the concentration of active Cas13 in the system.

Fluorescence curves were converted to activity scores by fitting the curves to effectively first-order reactions. With a certain amount of active Cas13, the concentration of uncleaved reporter is expected to decrease exponentially according to the reaction E*+U→E*+P, where E* is the concentration of active Cas13, U the concentration of uncleaved reporter, and P the concentration of cleaved reporter RNA. Labeling the (second-order) rate constant of this reaction as r, the concentration of P changes over time according to P(t)=Pₜₒₜ−(Pₜₒₜ−P(0))e^{rEt}.

Assuming that E* is constant over time, an activity score v=rE* is defined, which is an effective first-order rate constant (with units of inverse time). Assuming that r is constant across these assays, the activity score v is thus a proxy for the amount of active Cas13. Given measured P(0), best-fit values of Pₜₒₜ and v are found to fit the kinetic curves. To account for curves very far from saturation (e.g. NTC data) a minimum value of Pₜₒₜ is set based on data from saturating and near-saturating curves. For tiling data, the first 50 timepoints (˜4 hours) are fit to discount occasional apparent noise appearing at very late times.

Some assays including crRNA occluders displayed fluorescence curves that did not fit well to this effective first-order reaction, indicating a need to relax the assumption that A is constant over time. In some instances, the first several timepoints measured (15 and 10 timepoints, respectively, corresponding to 75 and 50 min) were neglected, as it was found that doing so increased the goodness of fit. For other assays using crRNA occluders—and those assays being directly compared to them, data was fit to a series of two effective first-order reactions: U→I→P. Labeling the first-order rate constant of each reaction k₁ and k₂, this model yields

$$P ( t ) = {P}_{tot} - ( {P}_{tot} - P ( 0 ) ) \frac{{k}_{1} {e}^{- {k}_{2} t} - {k}_{2} {e}^{- {k}_{1} t}}{{k}_{1} - {k}_{2}} .$$

Activity was defined in this case as v=(1/k₁+1/k₂)⁻¹, verifying that if the equation is first order (i.e. k₁>>k₂), the previous definition of activity is recovered. Indeed, negligible change in the measured activities for no-occluder control (NOC) fluorescence curves was found between these two fits.

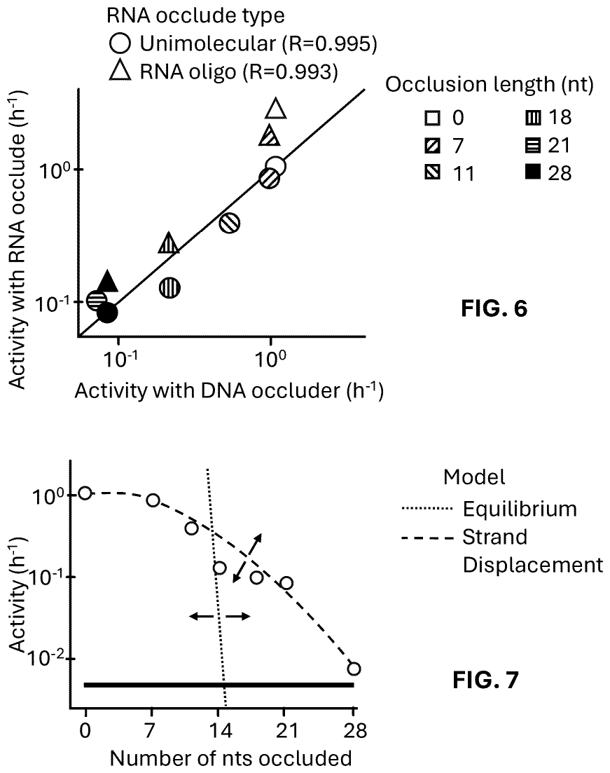

A high degree of correlation was observed between the three types of target occlusion. See FIG. 6. Cas13 activity varied by an order of magnitude for the same sequence with different amounts of target occlusion. See FIG. 7.

Similar variation in activity was also found as a result of RNA structure for the Cas13 orthologs LbuCas13a and RfxCas13d, as well as for LwaCas13a with a spacer sequence of length 21 nucleotides, demonstrating the generality of occlusion effects.

Activity reduction is quantitatively explained by a kinetic strand displacement model.

An equilibrium model based on the free energy of each target RNA failed to quantitatively account for the degree of structure-mediated Cas13 activity reduction (see FIG. 7, dotted line).

Labeling the free energy of the target-occluder complex by ΔGᵤ, the disagreement between the large difference in thermodynamic drives (exp[−βΔGᵤ] ranges over 30 orders of magnitude) and the smaller difference in activities (ranging over 2 orders of magnitude) cannot be explained by an equilibrium RNA-RNA hybridization framework. Given known free energies of RNA-RNA binding, the system temperature would have to be ˜7500 K to match an equilibrium model to the measured Cas13 activity reduction.

Therefore, a suitable alternative framework was sought to explain the measured activity levels. A strand displacement model presents one such framework. In this model, after initial binding to the target, the crRNA and occluding strand compete through a random-walk-like process until either the occluding strand is fully displaced or the crRNA-Cas13 complex dissociates from the target RNA. See FIG. 1.

It is hypothesized that strand displacement must occur for Cas13 to bind structured RNA. In this model, Cas13 first binds non-specifically to a region of RNA and then performs a local search for a sequence complementary to its bound crRNA. If Cas13 is not activated within a given time t_{dwell} (i.e. it does not fully bind to a protospacer sequence complementary to its crRNA) it dissociates from the RNA and repeats the search process. This sequence of events is analogous to the process by which enzymes such as the E. coli lac repressor search for their binding site on DNA. The strand displacement model predicts that as secondary structure length increases, the probability of Cas13 completing the strand displacement reaction within the time t_{dwell} decreases, leading to a proportional decrease in Cas13 activity. This model fits the measured results with a value of t_{dwell} equivalent to 100 steps of a strand displacement reaction (˜2×10⁻⁵s), in good agreement with direct measurements of typical dwell times for DNA-binding proteins (see FIG. 7, dashed line).

### Strand Displacement Model

In the strand displacement model, non-specific binding of Cas13 to the RNA (independent of RNA sequence) leads to Cas13 activation, and a corresponding decrease in Cas13 dissociation rate, when the crRNA fully binds to the protospacer complement. The typical dwell time of Cas13 on the RNA in the absence of this activation is denoted by t_{dwell}, the main parameter in the model. Secondary structure affects activity by modulating the probability that a strand displacement reaction completes within this time t_{dwell}. It is assumed that activity is directly proportional to this probability. To estimate this probability, for each occluder, 105 unbiased random walks of length equal to the number of occluded protospacer nucleotides with a reflecting boundary at 0 were simulated, measuring the number of steps taken to complete the random walk, and therefore the probability of completing the random walk within a desired number of steps. The number of trials chosen leads to errors in the estimate of this probability <3% in all cases (determined by the maximum ratio of standard deviation to the mean of our estimate across 10 replicates of 105 random walks).

To estimate a typical value of t_{dwell}, one can turn to classic studies of the E. coli lac repressor, a DNA-binding enzyme which searches for its binding site on the DNA by iteratively binding non-specifically to the DNA, performing a local search, and dissociating. The dissociation rate of the lac repressor when non-specifically binding DNA (i.e. the inverse of its dwell time) has been estimated to be 5×10⁴/s. This dwell time corresponds to the time it would take for ˜100 steps of a strand displacement reaction, where the rate of individual steps has been estimated to be 6×10⁷×e^{-2.5}/s≈5×10⁶/s. This dwell time varies with ionic concentration (among other factors), with dwell time decreasing anywhere from 2-10-fold upon doubling KCl concentration.

Given the different conditions used in the plate-reader assays and in the tiling experiments, including different ionic conditions—with the former having ˜2.5-fold higher KCl concentrations than the latter—t_{dwell} was fit separately for the two experimental methods. A dwell time of 100 steps of a strand displacement reaction was used for the plate-reader assays, and a dwell time of 300 steps for the tiling experiments.

To account for the asymmetry seen in various data, dwell time was allowed to change depending on whether the 5′ end of the protospacer was occluded or unoccluded. For the plate reader assays, the final model has a dwell time of 100 steps for those cases where the 5′ end of the protospacer was occluded, and a dwell time of 200 steps for those cases where it wasn't. For the tiling assays, the dwell times used are 300 and 600 steps, respectively.

### Tiling Experiment Activity Correction

Each experimental condition in the tiling experiment was performed with four replicates: two technical replicates for each of the two “shuffles” of the 961-nt-long target sequence. While excellent agreement was found between technical replicates, there was some variation between the results from each of the two target “shuffles”. This variation was apparent in and correlated between the positive controls of crRNAs 1 and 10, which were always unoccluded. It is hypothesized that this variability results from small variations in target concentration in our different samples. To correct for such variations, it was sought to quantify how much each RNA sample differed from the mean. The RNA samples were divided into 192 sample conditions, each corresponding to a single oligo pool and one target shuffling. Each of these 192 conditions was mixed with 24 assay conditions, corresponding to 8 experimental crRNAs, 2 positive control crRNAs, one non-targeting crRNA, one no-crRNA control (NPC) and two replicates of each.

For the two replicates of crRNAs 1 and 10 (i.e. for each of the 4 positive control assay conditions out of the 24 total assays), the activity fit from the mean fluorescence curve, averaging over the 192 sample conditions, was considered. Then, for each sample condition, and for each positive control, the ratio of the control's activity to its mean activity was calculated across all samples, obtaining an estimate of the degree to which that sample's concentration deviated from the mean. A correction factor may be defined as the average of these ratios. All activities measured for that sample were then divided by this correction. This activity correction not only decreased the spread of activities measured by the positive controls but also decreased the variance between measurements made on the two target “shuffles”.

### A Massively Multiplexed Assay Reveals Structure Effects Are Sequence Independent

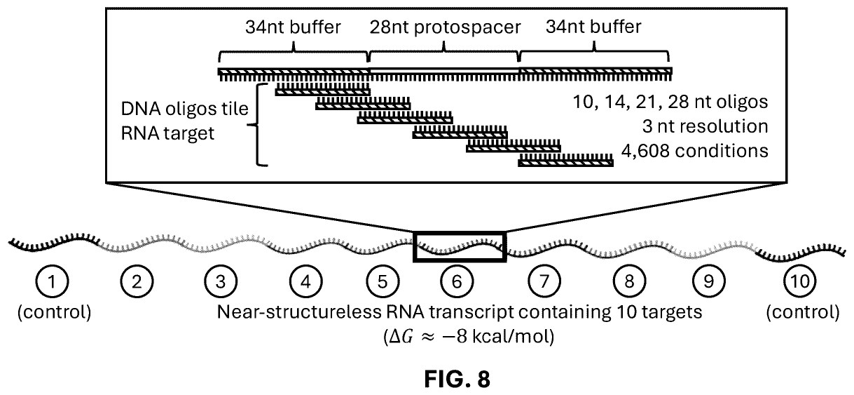

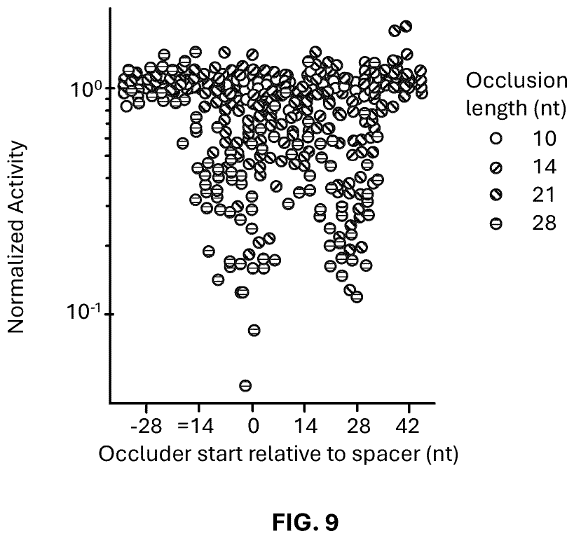

To probe the limits of the strand displacement model, a massively multiplexed assay was used to explore a broad range of target structure conditions for multiple sequences. The assay uses DNA oligos to create secondary structure at defined positions in the target, having previously validated their effect as a proxy for RNA structure (see FIGS. 4, 8). A single 1 kb-long RNA molecule was designed with minimal internal secondary structure. A NUPACK prediction estimates its minimum free energy due to intramolecular contacts to be ˜−6 kcal/mol, on par with random 35-nt-long RNA sequences. The target RNA is divided into two control blocks (one at each end of the molecule) and eight experimental blocks, allowing for efficient multiplexing. Each block contains a 28-nt-long protospacer flanked by two 34 nt buffer regions. For occlusion, DNA oligos of lengths 10, 14, 21, and 28 nucleotides (nt) were used in 3-nt-spaced tilings, for a total of 4,608 simultaneous conditions. These conditions were tested in parallel using a microfluidic chip-based assay. Summaries of the resulting dataset are shown in FIG. 9.

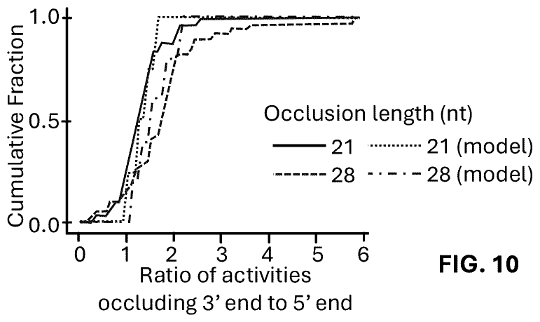

The results demonstrate that the reduction in Cas13 activity as a result of target structure is relatively sequence-independent, with the 8 experimental target blocks showing similar activity profiles in spite of large variation in absolute activity across these blocks. While 10- and 14-nt-long occluders had negligible effects on Cas13 activity, 21- and 28-mers had a strong effect. Consistent with earlier results, occluders binding to more of the protospacer typically led to a greater activity reduction. In agreement with the dwell time model and in contrast to other strand displacement systems, the presence or absence of toeholds (unoccluded RNA) had little effect on Cas13 activity. The data also revealed an unexpected asymmetry among the effect of occluders on Cas13, in which occluders binding to the 5′ end of the protospacer had a larger effect on Cas13 activity than occluders binding the same number of nucleotides at the 3′ end. See FIG. 10.

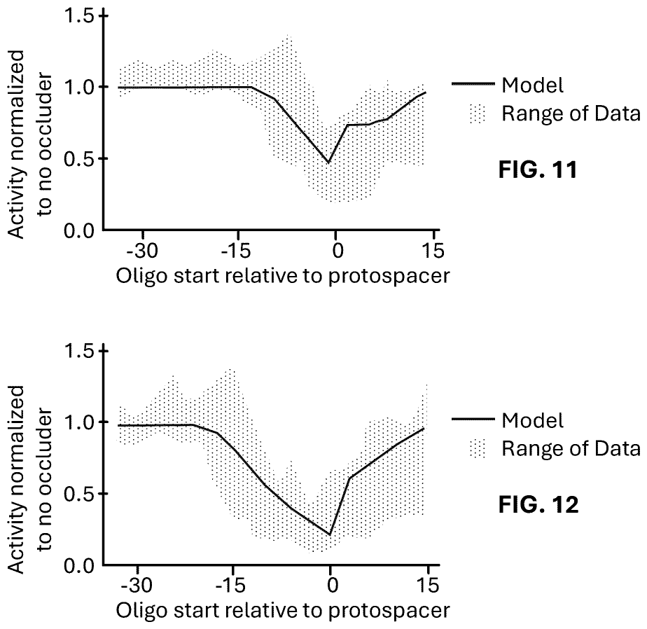

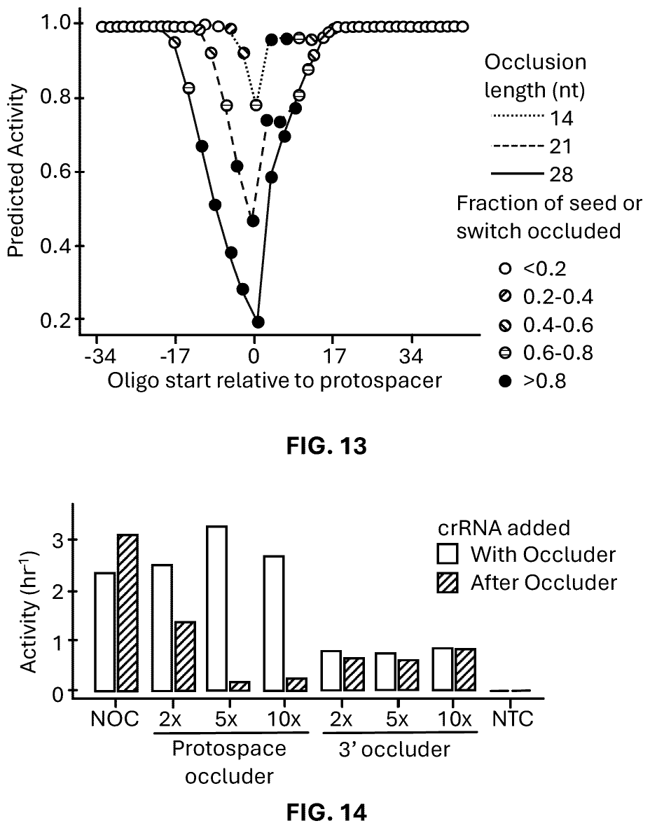

The asymmetry was accounted for in the model by including a second parameter in the model, creating a differential in t_{dwell} depending on whether or not the 5′ end of the protospacer is occluded, with binding preferentially initiating at the distal region of the protospacer, in agreement with previous findings. The revised dwell time model was able to quantitatively capture the effects of secondary structure on Cas13 activity (see FIGS. 10-12). This strand displacement model provides an alternate framework for prior results, demonstrating the importance of “seed” and “switch” regions at positions 5-8 and 9-14, respectively. See FIG. 13.

### Structure Occluding the Region 3′ of the Protospacer Inhibits Cas13

Surprisingly, when occluders are placed directly 3′ to the protospacer, Cas13 activity is potently inhibited. This second regime of inhibition exists across all tested crRNAs, and inhibition is strong for both 21 mer and 28 mer occluders. The non-monotonicity of this second activity trough cannot be explained using a strand displacement model, implying this drop in activity is not due to a reduction in crRNA-target binding. In agreement with this hypothesis, an electrophoresis mobility shift assay (EMSA) showed that 3′ occlusion leads to negligible reduction in binding affinity of the crRNA-Cas13 complex to the target, as opposed to protospacer occlusion which leads to a significant reduction.

To probe more fully whether the effect of occluders on Cas13 activity is a result of competitive or allosteric inhibition, the typical protocol of pre-annealing occluders to the target was modified. For the protospacer occluder, but not for the 3′ occluder, full rescue of Cas13 activity was observed when the crRNA and occluder are added at the same time. Increasing the concentration of 3′ occluder does not increase its inhibitory effect (see FIG. 14). These results indicate that the activity reduction conferred by occluding the region 3′ to the protospacer is likely the result of an allosteric rather than a competitive inhibitory effect.

### Strand Displacement Enhances Mismatch Detection

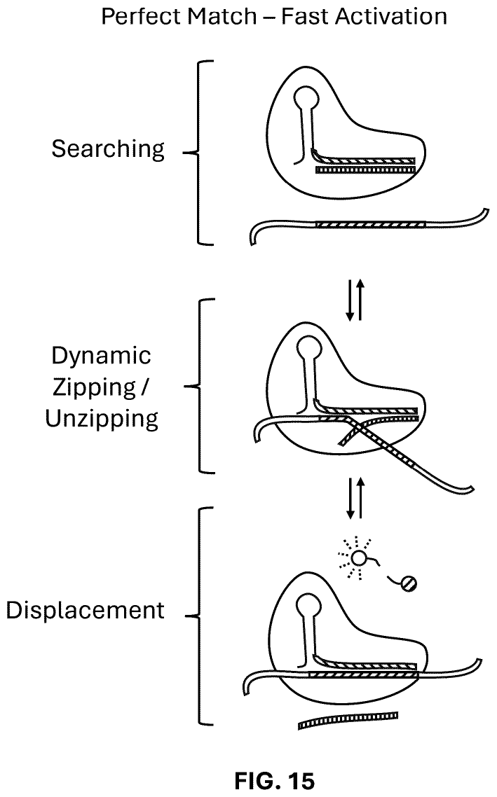

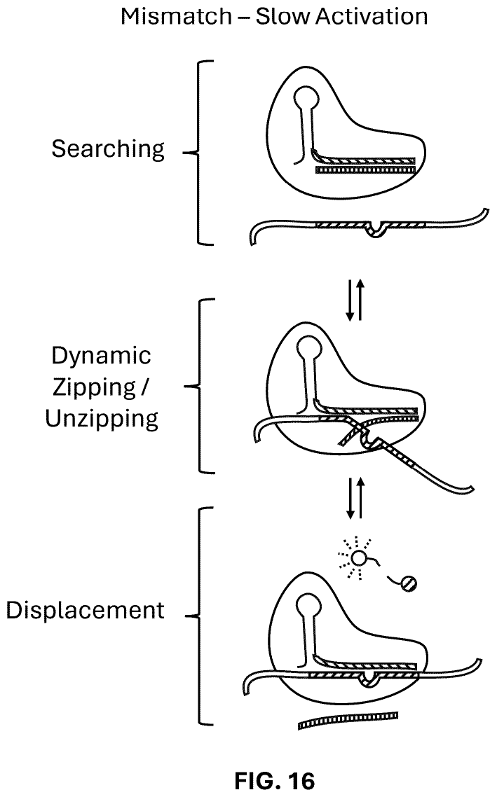

It is hypothesized that the insights from the strand displacement model could help dramatically improve the specificity of Cas13-based RNA detection assays. Past work has shown that secondary structure can make nucleic acid hybridization more sensitive to mismatches, both in CRISPR-based approaches and in other assays; it is hypothesized that given the kinetic nature of the disclosed assays, one could leverage the kinetic nature of strand displacement to similar ends without the necessity of a binding toehold required in other approaches. With no internal structure, even a mismatched crRNA is expected to bind strongly to the target in the disclosed model; however, an occluding strand provides an extra kinetic barrier that is less likely to be overcome by a mismatched crRNA than by one that is perfectly complementary, thus improving specificity given the short dwell time of inactive Cas13 on the RNA. See FIGS. 15-16.

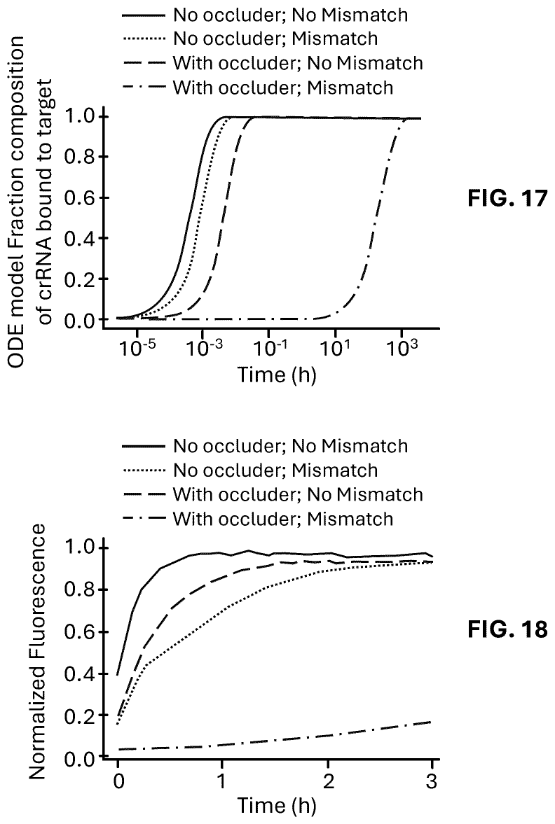

A differential-equation-based strand displacement model (ODE model) supports this hypothesis, revealing that even when both complementary and mismatched invader strands bind strongly to the target in equilibrium, the mismatched invader takes much longer to bind than the perfectly-matched invader in the presence of an occlude. See FIG. 17.

For the ODE model, the binding rate of an invading strand was compared to a target with and without an occluder in a model based on Srinivas & Oulridge et al. (2013) and Irmisch et al. (2020). The model consists of a set of ordinary differential equations (ODEs) representing the flux into and out of states, where each state is defined by the set of base pairs formed. Transitions between states occur at a rate ke^{-ΔG} where ΔG is the free energy barrier to the transition (in units of k_{B}T where k_{B} is Boltzmann's constant and T is temperature in units of Kelvin) and k is an overall rate constant. An initial state consists of a target strand (bound to an occluding strand in the case where an occluding strand is considered), with an invading strand unbound. Initial binding of the invader strand to the toehold has a free energy barrier of ΔGₐ. The reverse step has a barrier of hΔG_{R} where h is the toehold length and ΔG_{R} is the (absolute value of the) typical free energy of an RNA-RNA base pair.

In the no-occluder case, subsequent forward steps (in which an additional base pair between target and invader forms) have a free energy barrier of 0, while reverse steps have a free energy barrier of ΔG_{R}.

In the occluder case, the first step of the strand displacement reaction has barrier ΔG_{P}+ΔG_{S}−(ΔG_{R}−ΔG_{D}), where (ΔG_{R}−ΔG_{D}) has been subtracted from the models on which these examples are based to account for the fact that in the example system, the invading strand is RNA while the occluding strand is DNA; ΔG_{D} is the (absolute value of the) typical free energy of an RNA-DNA base pair. Subsequent forward steps in the strand displacement reaction have a barrier of ΔG_{S}−(ΔG_{R}−ΔG_{D}), while backward steps all have a barrier of ΔG_{S}. The barrier from the final state, in which the occluder has fully dissociated, back to the penultimate state, has a barrier of ΔG_{DD}.

Parameters were set following Irmisch et al. to: ΔGₐ=18.6; ΔG_{R}=2.52; ΔG_{S}=7.4; ΔG_{P}=3.5; ΔGₘ=9.5 (all in units of k_{B}T); and an overall rate constant of k=6×10₇/s. ΔGD is set equal to 1.2 to be roughly half of ΔG_{R}, and ΔG_{DD}=25 to be large enough to prevent reassociation on the timescales considered. In FIG. 17, the results of h=3, b=27, with a mutation at the first position after the toehold, are plotted.

Cas13′s ability to differentiate between a perfectly complementary target and one containing a single A>U mutation at position 5 of the protospacer with and without secondary structure occlusion was tested. Both occluding the target and occluding the crRNA was tested, reasoning that strand displacement would in either case result in improved mismatch discrimination. It was decided to focus on crRNA occluders as these provide the added benefit of improving mismatch detection regardless of the identity of the mismatched target, and their use does not require any sample manipulation prior to detection. The presence of a crRNA occluder resulted in a ˜50×enhancement of specificity compared to the no-occluder condition, measured as the maximum ratio of WT/mismatch fluorescence (see FIG. 18). This effect was robust to large variations in target concentration, and was maximized at higher ˜1-100 nM target input concentrations. See

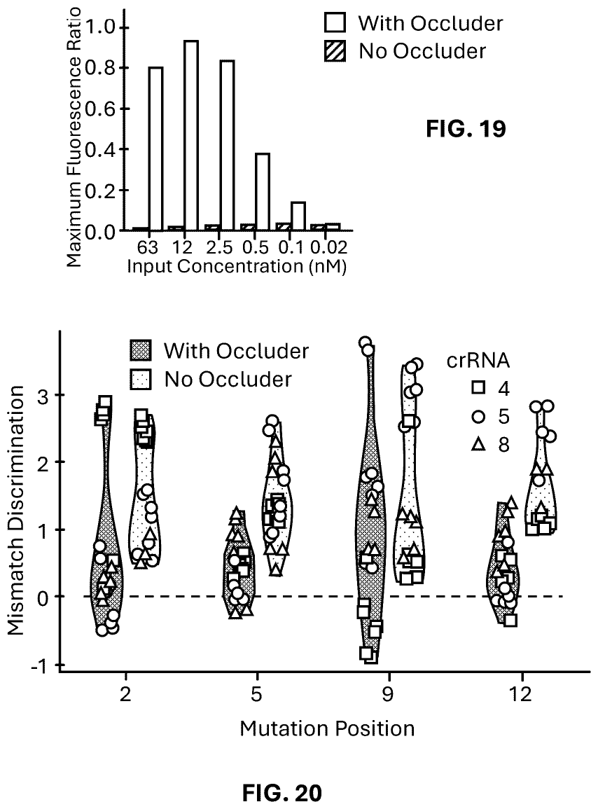

FIG. 19 shows the results of one simple metric by which to measure mismatch discrimination: the maximum of the ratio FPM/FMM, where FPM is the average fluorescence measurement across the perfectly matched conditions, and FMM across the mismatched conditions. To account for the arbitrary offset of fluorescence, the minimum fluorescence measured across the NTC experiments was subtracted from both FPM and FMM before taking the ratio. Since FPM and FMM are each measured as the average across three replicates, each measurement of FPM and FMM has an inherent error, σPM and σMM (respectively), which was quantified as the standard deviation across the three replicates at each timepoint. The error of the ratio is then propagated as \sqrt{(F_{PM}σ_{MM})²+(F_{MM}σ_{PM})²}/(F_{MM})².

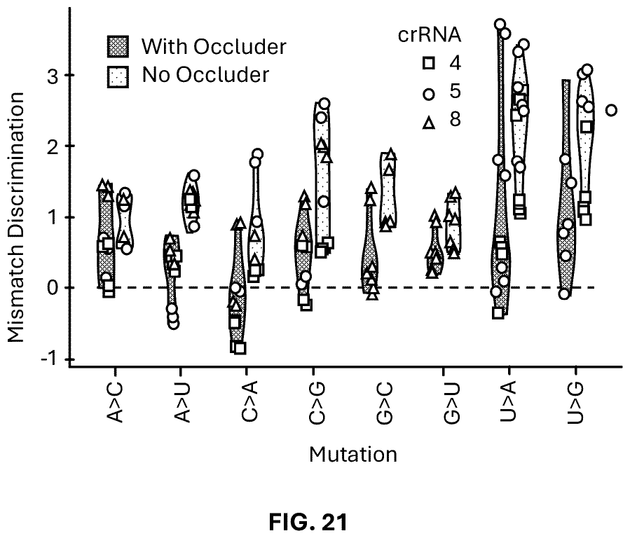

The generality of Cas13 specificity enhancement by occluders was then explored. Using 3 different targets, 4 positions on each crRNA, 2 mutations for each position, and 4 replicates, how well a mismatch could be detected by Cas13 with and without a crRNA-occluder was tested. Cas13 activity was measured on the perfectly matched and mismatched targets, finding that although only 74/96 mismatches (77%) led to any activity reduction in the absence of a crRNA-occluder, all 96 (100%) led to a reduction with the occlude. See FIGS. 20-21.

For FIGS. 20-21, mismatch discrimination was measured by a metric that relies on activity fits: log₂(v_{PM}/v_{MM}). Thus, a mismatch discrimination of 1 indicates that the measured activity of the perfectly matched conditions is twice that of the mismatched conditions, and a discrimination of 3 indicates the perfectly matched conditions had eight-fold higher activity than the mismatched conditions.

Of the 24 separate mismatches tested, significant discrimination fails in 46% (11/24) without occluders, and in 0/24 with occluders (p>0.05, one-sided t-test). It was found that without occlusion, the ability of Cas13 to distinguish between a perfectly-matched target and a mismatched target is not guaranteed for any crRNA, for any of the mismatch positions that was tested, nor for any specific type of mutation (with the possible exception of G>U, for which the fewest data points were collected). However, using occluded crRNAs, Cas13 can distinguish perfectly-matched from mismatched targets in a position-and mutation-independent manner.

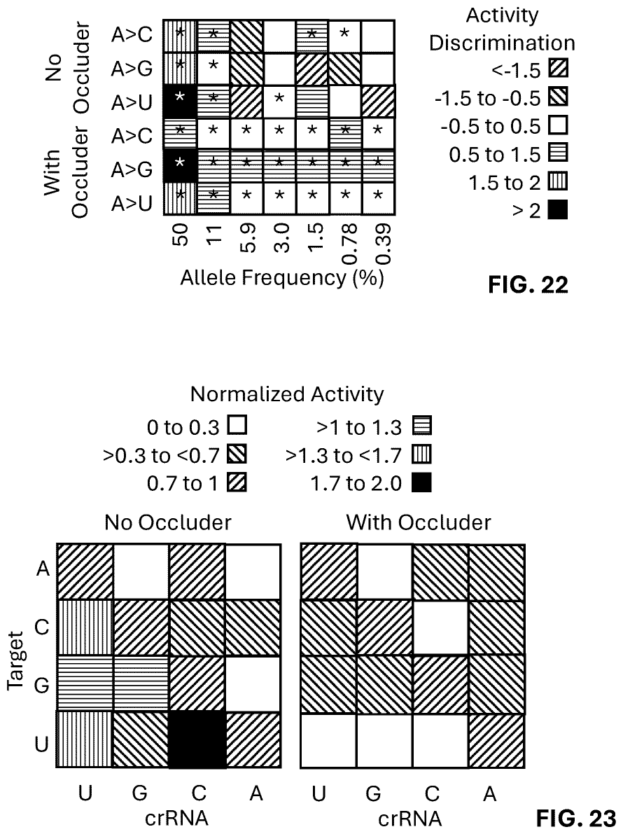

A dilution series of perfectly matched target RNAs was performed, then spiked this target RNA into solutions of single-mismatch off-target RNAs. Without occlusion, the perfectly matched target was detected at allele frequencies of 11% (a 1:8 ratio) but not 6% (1:16); with occlusion, it was detected at frequencies as low as 0.4% (1:256) for all tested targets, an order-of-magnitude sensitivity enhancement. See FIG. 22, where a similar measure to that for FIGS. 20-21 was used for discrimination at low allele frequencies, defining activity discrimination as log₂(v_{f}/v_{O}) where v_{f} is the activity measured at allele frequency f, and v_{O} the activity measured in the background alone.

To explore nucleotide-specific effects, we mutated the crRNA and target sequences to all possible nucleotides at position 5 of the spacer. When testing all pairwise crRNA/target combinations, we observed extensive cross-reactivity between crRNAs and targets in the absence of occlusion. With occlusion, we observed specific detection of each target only by its perfect-match crRNA. See FIG. 23. Occluded crRNAs can thus discriminate all four possible alleles at a given position, demonstrating the exquisite specificity of the approach.

### Occluders Enable Variant Calling in Diagnostic Settings

To test the efficacy of our occlusion strategy in real-world diagnostic contexts, a set of SARS-CoV-2 and influenza A virus (IAV) variants of clinical significance was considered, and the extent to which Cas13 can distinguish among these both with and without occluders was measured.

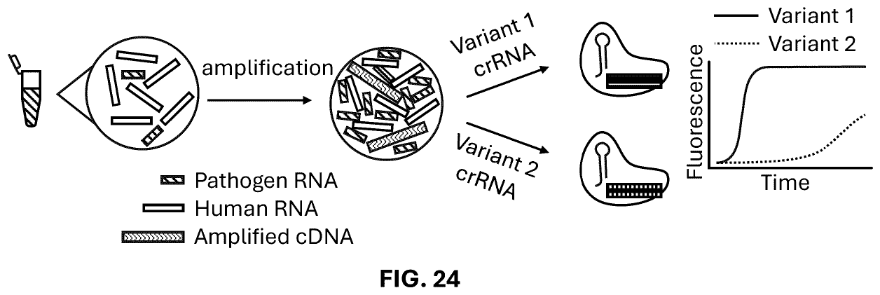

While most crRNA spacers disclosed herein were designed to be perfectly complementary to their 28 nucleotide (nt) protospacer region, for SARS-CoV-2 targeting sequences, a single synthetic mismatch was inserted at position 5 to improve baseline specificity. Spacers were appended to the 3′ end of the consensus direct repeat sequence for the Cas13 ortholog being employed, and ordered from IDT as Alt-R guide RNA. For example, cr4_Mut5(U>C)crRNA used a direct repeat sequence of GAUUUAGACUACCCCAAAAACGAAGGGGACUAAAAC (SEQ ID NO. 1) and a spacer sequence of CAUGCGUGGGGAGUUCUUUGAUGGCAAC (SEQ ID NO. 12). A different spacer variant, instead of a U>C mutation, had a U>G mutation. See FIG. 24.

While testing various occluder/target pairs, we found that a small subset of DNA sequences can serve as targets for LwaCas13a, activating trans-cleavage activity, a finding that has recently been reported by others for LbuCas13a36. To mitigate this effect, several modified occluders were tested, finding that incorporating a single locked nucleic acid (LNA) at position 16 of the occluder (measured relative to the spacer) eliminates background activity and does not compromise performance.

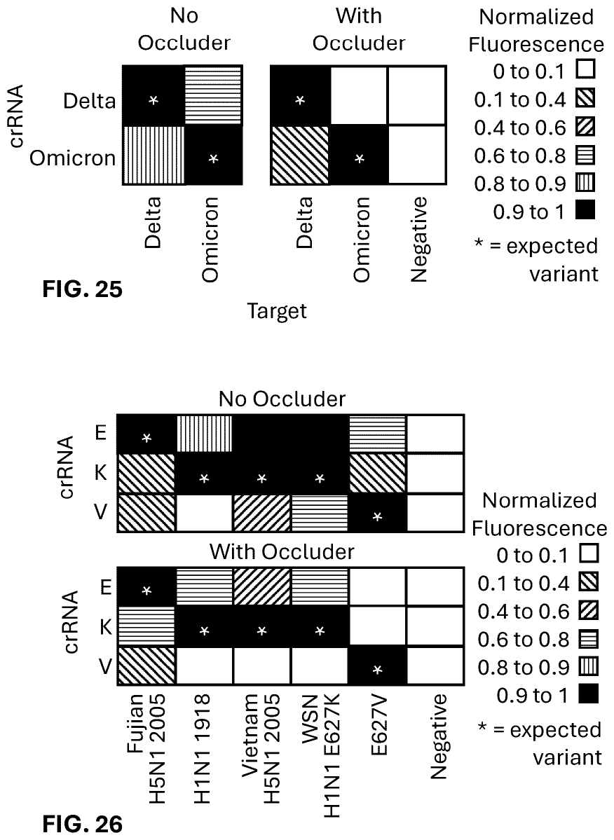

It was first sought to diagnose SARS-CoV-2 variants in amplified viral seedstocks, finding that occluded Cas13 is able to distinguish between the B.1.617.2 (Delta) and B.1.1.529 (Omicron) variants. See FIG. 25 and Table 2, below.

TABLE 2
(Delta and Omicron information)

| Description | Delta | Omicron |
|---|---|---|
| crRNA Direct Repeat | GAUUUAGACUACCCCAA | GAUUUAGACUACCCCAA |
| Sequence | AAACGAAGGGGACUAA | AAACGAAGGGGACUAA |
| | AAC (SEQ ID NO 1) | AAC (SEQ ID NO 1) |
| crRNA Spacer Sequence | CAAGCUUUGCUACCGGC | CAAGCUUUGUUACCGGC |
| | CUGAUAGAUUU (SEQ ID | CUGAUAGAUUU (SEQ ID |
| | NO 13) | NO 14) |
| Target Spike gene | GAAATTAATACGACTCA | GAAATTAATACGACTCA |
| | CTATAGGGAAGTCTAAT | CTATAGGGAAGTCTAAT |
| | CTCAAACCTTTTGAGAG | CTCAAACCTTTTGAGAG |
| | AGATATTTCAACTGAAA | AGATATTTCAACTGAAA |
| | TCTATCAGGCCGGTAGC | TCTATCAGGCCGGTAAC |
| | AAACCTTGTAATGGTGT | AAACCTTGTAATGGTGT |
| | TGAAGGTTTTAATTGTTA | TGCAGGTTTTAATTGTTA |
| | CTTTCCTTTACAATCATA | CTTTCCTTTACGATCATA |
| | TGGT (SEQ ID NO 15) | TAGT (SEQ ID NO 16) |
| Occluder Spacer | AAATCTATCAGGCCGGT | AAATCTATCAGGCCGGT |
| Sequence | AGCAAACCTTG (SEQ ID | AACAAACCTTG (SEQ ID |
| | NO 17) | NO 18) |
| Primer (Rv) | ATGATTGTAAAGGAAAG | ATGATCGTAAAGGAAAG |
| | TAACAATTAAAACCTTC | TAACAATTAAAACCTGC |
| | AACACC (SEQ ID NO 19) | AACACC (SEQ ID NO 21) |
| Primer (FwT7) | TAATACGACTCACTATAg | TAATACGACTCACTATAg |
| | ggCTCAAACCTTTTGAGA | ggCTCAAACCTTTTGAGA |
| | GAGATATTTCAAC (SEQ | GAGATATTTCAAC (SEQ |
| | ID NO 20) | ID NO 22) |

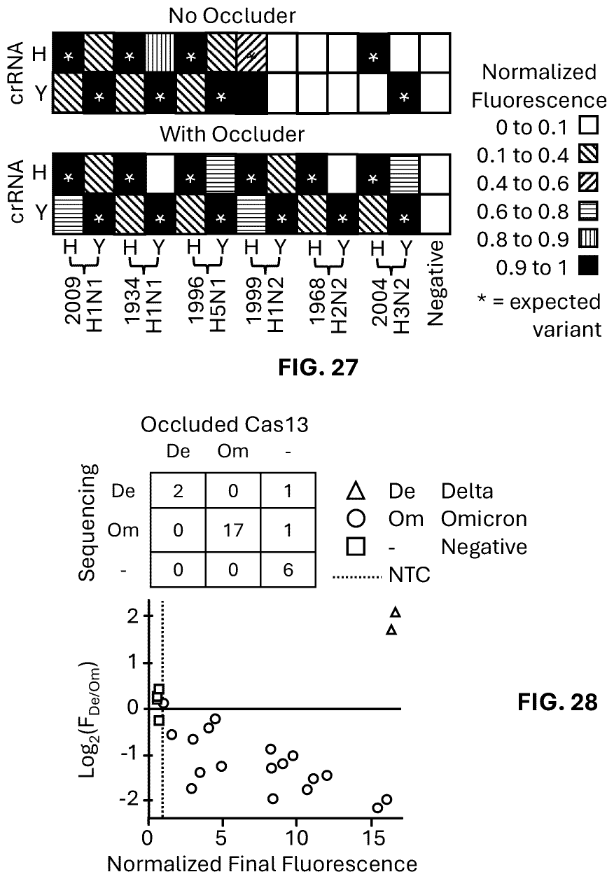

Next, IAV variants with public health relevance were tested. Of particular concern is the E627K mutation in the PB2 gene of avian IAV strains, a single nucleotide change associated with mammalian adaptation which increases avian IAV replication and pathogenicity in humans and which can currently only be diagnosed by sequencing. With occluders, Cas13 was able to robustly distinguish the ancestral (627E) variant from multiple mammalian-adapted (627K) strains, as well as the 627V variant, which is becoming increasingly prevalent. See FIG. 26. Another clinically important mutation occurs in the NA gene, where a single nucleotide H to Y conversion (commonly at position 275 in seasonal N1 strains) confers resistance to oseltamivir (Tamiflu). This H>Y mutation was introduced into various strain backgrounds (1934 H1N1, 1968 H2N2, 1996 H5N1, 1999 H9N2, 2004 H3N2, and 2009 H1N1). Occluded Cas13 distinguished the WT from the mutant variant in all strains using only a single guide pair per NA subtype (i.e. one for N1 strains, one for N2 strains). See FIG. 27.

To realize real-world deployability, the disclosed occluder methodology was integrated into SHERLOCK, a multiplexed and portable Cas13-based RNA detection protocol, finding that occluder-enhanced detection displays sensitivity of 10 cp/uL, the lowest concentration tested.

26 SARS-CoV-2 patient samples from the United States (20 positive and 6 negative samples) were tested. Occluded Cas13 was able to robustly distinguish positive from negative samples, and to distinguish Delta from Omicron variants, failing to detect only a single sample with a Ct value>35 and making no incorrect calls. See FIG. 28, 95% sensitivity, 100% specificity.

To perform discrimination, the fluorescence ratio between crRNAs targeting the variants at the time where this ratio was highest was measured, labeling the ratio F_{v1/v2} for variants v₁ and v₂.

For these field-deployable results, a method of activity discrimination was implemented that did not require curve-fitting. This method has two steps: 1) determine whether the sample is positive or negative for the RNA in question; 2) if positive, determine which variant is present.

For step #1, whether the maximum fluorescence reached is higher than the fluorescence of the negative “no target control” (NTC) was tested. To avoid false positives, the maximum NTC value was used, and to account for NTC variability, inflated this number slightly when considering patient samples (multiplying it by 1.7). When two NTC conditions were assayed, one was used to determine this cutoff (and for normalization in step 2) and the other was plotted.

Having determined which samples were positive, we then proceeded to discriminate among the variants. When more than two variants were present (see, e.g., FIGS. 26, 32), it was ensured the assays did not saturate and thus one was able to use final fluorescence values (at 180 minutes) as a measure of activity.

$$( F ( c , t ) - m ) / ( {max}_{c} F ( c , t ) - m )$$

was plotted, where F(c, t) is the final fluorescence of crRNA c detecting target t, and m is the maximum NTC value. In FIG. 32, m was set to be the minimum fluorescence value of each target since the fluorescence curves were very far from saturation and relatively close to NTC.

When only two variants were being discriminated between (e.g., FIGS. 25, 27-30, 31), the fluorescence values at which discrimination was highest in place of the final fluorescence values in the above analysis. This enabled one to measure discrimination even for targets where both variants' crRNAs saturated within the assay's 180 minutes. To this end, for each target, the ratio of fluorescence time-series were considered for the two crRNAs, F_{v1/v2}(t). The logarithm of this curve was then taken, so that positive values imply that detection by one crRNA was higher, and negative numbers imply that detection by the other crRNA was higher. The timepoint at which the absolute value of this number was maximal was focused on, with the caveat that for each target, timepoints for which the maximum fluorescence across crRNAs was below the NTC threshold was neglected. In FIGS. 25, 27, and 31, the fluorescence of each crRNA at this timepoint is shown, and plotted as in the other figures as

$$\frac{F ( c , t ) - m}{{max}_{c} F ( c , t ) - m}$$

but where here, F(c, t) is the fluorescence at the timepoint that maximizes discrimination. In FIGS. 28-30, the maximum fluorescence reached for each target was plotted on the x-axis, and the maximum discrimination (which is denoted log₂ F_{v1/v2}) on the y-axis.

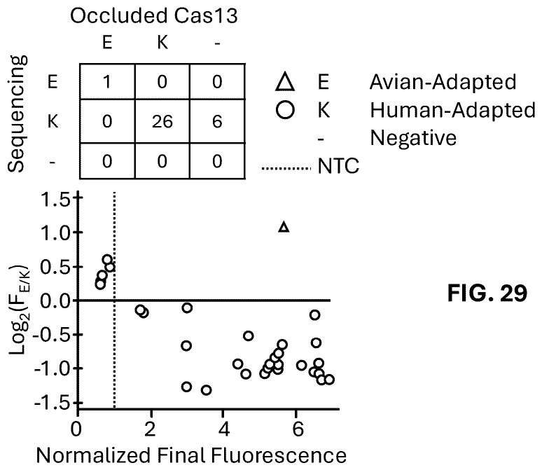

33 seasonal H1N1 or H3N2 IAV positive patient samples from the United Kingdom were also analyzed and it was confirmed that occluded Cas13 was able to correctly identify the 627E or K variant in all samples for which Cas13-based detection showed a positive signal; 6 patient samples tested negative due to poor amplification resulting from sequence variation in the primer binding regions. See FIG. 29, 82% sensitivity, 100% specificity.

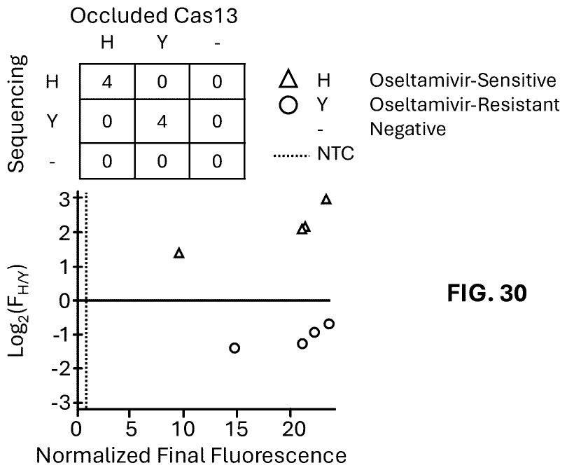

8 IAV A(H1N1)pdm09 samples were tested from infected patients from the Netherlands for the single-nucleotide oseltamivir-resistance mutation NA H275Y, correctly calling all samples as containing the wildtype or resistant variant. See FIG. 30, 100% sensitivity, 100% specificity. Moreover, occluded Cas13 was able to distinguish Delta from Omicron strains in patient samples using a visual fluorescent readout.

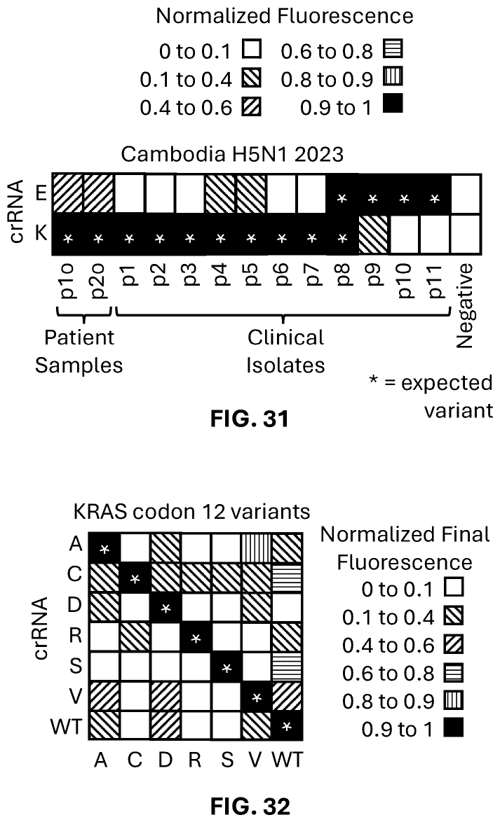

In the midst of the ongoing H5N1 avian influenza outbreak, our E627K assay was deployed for variant surveillance in Cambodia and 2 patient samples and 11 clinical isolates from patients who had tested positive for H5N1 were tested. Occluded Cas13 was able to robustly distinguish the patients with the E variant from those with the K variant with 100% sensitivity and specificity. See FIG. 31. Additionally, the occluded Cas13 assay called one clinical isolate (which was originally reported to be the avian-adapted E variant) as being positive for both E and the mammalian-adapted K. Upon deep sequencing, this isolate was indeed confirmed to contain both E (50.8%) and K (48.3%) variants at position PB2 627. This underlines the power of occluded Cas13 in detecting rare or minor variants which may be missed in sequencing-based assays.

The variability of Cas13′s specificity has so far hampered the potential of employing Cas13 for mutation detection at scale. The disclosed occluder methodology is therefore poised to expand the utility of Cas13 for SNP detection beyond viral diagnostics.

To demonstrate this principle, occluded Cas13 was employed to distinguish somatic variants in the KRAS gene, a pan-cancer oncogene mutated in over 20% of human cancers. 7 somatic variants of codon 12 were focused on, as this site is highly polymorphic and represents over 90% of oncogenic KRAS mutations.

Its mutants are associated with negative outcomes for cancer patient survival, though different mutations have differential prognoses and treatment options, highlighting the importance of correct variant diagnosis. In order to multiplex this large-scale panel, occluders were integrated into mCARMEN, which leverages microfluidics to test a large number of samples for a panel of crRNAs simultaneously.

Occluded Cas13 was able to robustly distinguish all 7 KRAS variants from one another, even though 24 out of 42 variant pairs are distinguished by only a single-nucleotide substitution and none are distinguished by >2 substitutions. See FIG. 32.

In the examples discussed herein, RNA targets were ordered from Integrated DNA Technologies as DNA containing a T7 promoter sequence. Targets were then transcribed to RNA using the T7 HISCRIBER® High Yield RNA Synthesis Kit in 55 μL reactions (New England Biolabs) with a 16 h incubation step at 37° C. and purified with 1.8× volume AMPure XP beads (Beckman Coulter) with the addition of 1.6× isopropanol, then eluted into 20 μL of nuclease free (NF) water. All RNAs were then quantified using a NanoDrop One (Thermo Fisher Scientific) or Biotek Take3 Trio (Agilent) then stored in nuclease free (NF) water at −80° C. for later use.

Occluded targets and crRNAs were prepared by mixing DNA/RNA oligo occluders with target RNA or crRNA in 60 mM KCl (Invitrogen) in NF water at a ratio of 2:1 (BioMark assays) or 10:1 (plate reader assays) and put through an annealing cycle consisting of a high-temperature melting step at 85° C. for three minutes followed by gradual cooling to 10° C. at 0.1° C./sec followed by cooling to 4° C. For massively multiplexed assays, occluders were first pooled by length and start position within the target block such that each resulting oligo pool contained all 8 n-mers binding to a given position within each of the experimental target blocks. Targets and crRNAs were then used for detection assays immediately as described herein.

Targets were input into detection reactions at various concentrations. For experiments related to FIGS. 3-7, targets were input at 7.5×10⁸ copies/μL (cp/μL). For experiments related to FIGS. 8-13, targets were input at 8×10⁸ cp/μL. For experiments relating to FIG. 14, targets were input at 5×10⁹ cp/μL. For experiments relating to FIGS. 15-21, input concentrations of 7.5×10⁹ cp/μL were used unless otherwise noted in the figure description. For experiments related to FIG. 22, targets were spiked in at the indicated allele frequency into a background of 5×10¹⁰ cp/μL (for occluded conditions) or 5×10⁸ cp/μL (for non-occluded conditions). For experiments related to FIG. 23, occluded conditions used an input concentration of 5×10¹⁰ cp/μL while non-occluded conditions used a concentration of 5×10⁸ cp/μL.

Target controls were amplified from plasmids. Extracted viral genomic RNA samples were acquired from BEI Resources (hCoV-19/USA/MD-HP05285/2021 (B.1.617.2) Delta, hCoV-19/USA/GA-EHC-2811C/2021 (B.1.1.529) Omicron). Amplification reactions using 1 or 2 μL of viral RNA as the input (50 μL total reaction volume) were performed using the Qiagen One-Step RT-PCR kit according to the manufacturer's specifications.

Total RNA was extracted from clinical samples using the trizol-chloroform method. Extracted RNA was then amplified using QIAGEN One-Step RT-PCR (UK seasonal influenza samples, US SARS-CoV-2 samples, Cambodia H5N1 samples and isolates) or RT-RPA (TwistDx) (Netherlands seasonal influenza samples, select SARS-CoV-2 samples) using either 1 μL or 2 μL of input material.

What is claimed:

1. A method for enhanced nucleic acid detection, comprising:
providing a DNA or RNA oligonucleotide complementary to either a target RNA molecule or a crRNA spacer sequence, the crRNA spacer sequence being a reverse compliment of a target region of the target RNA molecule;
annealing the DNA or RNA oligonucleotide to the target RNA molecule or the crRNA spacer sequence by mixing the DNA or RNA oligonucleotide in excess with the target RNA molecule or the crRNA spacer sequence in an aqueous mixture at a first temperature;
ramping a temperature down from the first temperature to a second temperature at a first rate;
adding the aqueous mixture to one or more Cas13 RNA detection reagents; and
monitoring a fluorescent signal for a period of time.

2. The method of claim 1, wherein a ratio of DNA:RNA in the aqueous mixture is 2:1-10:1.

3. The method of claim 1, wherein the first temperature is 85° C.

4. The method of claim 3, wherein the second temperature is 4° C.

5. The method of claim 1, wherein the aqueous mixture comprise a salt.

6. The method of claim 5, wherein the salt is KCl.

7. The method of claim 1, wherein the one or more Cas13 RNA detection reagents is a mixture comprising water, an RNAse inhibitor, Leptotricia wadeii Cas13a (LwaCas13a), a detection buffer, a reporter RNA, crRNA, and magnesium acetate.

8. The method of claim 1, wherein the period of time is at least 10 minutes.

9. The method of claim 1, wherein the period of time is less than 6 hours.

10. The method of claim 1, wherein the DNA or RNA oligonucleotide is perfectly complementary to either the target RNA molecule or the crRNA spacer sequence.

11. The method of claim 1, wherein the DNA or RNA oligonucleotide is partially complementary to either the target RNA molecule or the crRNA spacer sequence.

12. The method of claim 1, wherein the DNA or RNA oligonucleotide is chemically modified.

13. A composition of matter comprising:
a Cas13 protein;
a CRISPR RNA (crRNA);
a target RNA molecule;
a reporter RNA; and
one or more occluders.

14. The composition of matter of claim 13, wherein the composition of matter consists of the Cas13 protein, the CRISPR RNA (crRNA), the target RNA molecule, the reporter RNA, and the one or more occluders.

15. The composition of matter of claim 13, wherein the one or more occluders is a DNA oligonucleotide, a RNA oligonucleotide, a hairpin extension of the crRNA, or a combination thereof.

16. The composition of matter of claim 13, wherein the Cas13 protein is Cas13, LwaCas13a (C2c2), LbuCas13a, PsmCas13b, PspCas13b, CcaCas13b, CasRx, Cas13d, orthologs thereof, or a combination thereof.

17. The composition of matter of claim 13, wherein the occluder is 5 nt to 50 nt in length.

18. The composition of matter of claim 17, wherein the occluder is 10 nt to 40 nt in length.

19. The composition of matter of claim 18, wherein the occluder is 15 nt to 35 nt in length.

20. The composition of matter of claim 19, wherein the occluder is 20 nt to 30 nt in length.

21. A method for improving crRNA design, comprising:
using a strand displacement model of Cas13 reaction kinetics as a function of secondary structure to identify a target crRNA sequence that achieves desirable reaction kinetics; and
producing crRNA having the target crRNA sequence.
